(19) United States
(12) Patent Application Publication
(10) Pub. No.: US 2006/0040951 A1
(43) Pub. Date: Feb. 23, 2006

(54) Use of 5-ht2 receptor antagonists for the treatment of sleep disorders

(75) Inventors: Gerd Bartoszyk, Weiterstadt (DE); Christoph Van Amsterdam, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Merck Patent GmbH, Darmstadt (DE)

(21) Appl. No.: 10/530,051

(22) PCT Filed: Sep. 3, 2003
(86) PCT No.: PCT/EP03/09738

§ 371 Date: Apr. 4, 2005

(30) Foreign Application Priority Data
Oct. 4, 2002 (DE) 102 46 357.3

(51) Int. Cl. A61K 31/496

(52) U.S. Cl. 514/254.09

(57) ABSTRACT

The invention relates to the use of 5-HT₂ receptor antagonists for the preparation of a medicament for extending both non-REM sleep an REM sleep.

The invention relates to the use of 5-HT₂ receptor antagonists for the preparation of a medicament for extending both non-REM sleep and REM sleep.

Novel N-(indolecarbonyl)piperazine derivatives and processes for the preparation thereof are disclosed in WO 01/07435. While being well tolerated, the substances exhibit, inter alia, actions on the central nervous system and has valuable pharmacological properties. They have strong affinity to 5-HT_{2A} receptors and have 5-HT_{2A} receptor-antagonistic properties.

WO 01/07435 furthermore discloses that the said N-(indolecarbonyl)-piperazine derivatives are suitable both in veterinary and in human medicine for the treatment of functional disorders of the central nervous system and of inflammation. They can be used for the prophylaxis and the combating of the consequences of cerebral infarction (apoplexia cerebri), such as strokes (here, for example, trauma) and cerebral ischaemia, and for the treatment of extrapyramidal-motor side effects of neuroleptics (for example dystonic syndrome, of muscle stiffness induced by neuroleptics, tremor (including substance-induced tremor forms) or extrapyramidal movement disorders), and of Parkinson's disease, including dopaminomimetic side effects of conventional Parkinson's medicaments, for the acute and symptomatic therapy of Alzheimer's disease and for the treatment of amyotrophic lateral sclerosis. The substances are likewise suitable as therapeutic agents for the treatment of brain trauma (for example after head injuries) or spinal cord trauma. However, they are particularly suitable as medicament active ingredient for anxiolytics, antidepressants, antipsychotics, neuroleptics, antihypertonics and/or for positively influencing obsessive-compulsive disorder (OCD), including anancastic spectrum disorders (obsessive-compulsive spectrum disorders, OCSD), anxiety states, panic attacks, psychoses, schizophrenia, anorexia, delusional obsessions, agoraphobia, migraine, sleep disorders, including sleep apnoea, tardive dyskinesia, learning disorders, age-dependent memory disorders, eating disorders, such as bulimia, drugs misuse (including disorders induced by substance abuse) and/or sexual dysfunctions.

They are furthermore suitable for the treatment of endocrinic diseases, such as hyperprolactinaemia, furthermore in vasospasms, hypertension, gastrointestinal diseases, cardiovascular diseases and extrapyramidal symptoms, as described in WO 99/11641 on page 2, line 24-30. In addition, the N-(indolecarbonyl)piperazine derivatives are suitable for lowering the intraocular pressure and for the treatment of glaucoma.

Further uses of these N-(indolecarbonyl)piperazine derivatives are described in WO 03/045392: thus, the substances are also suitable for the treatment of obesity, sub-types of anxiety, sub-types of schizophrenia and types of dementia of various origin and for the therapy of aggression disorders, Parkinson's disease, attention deficit disorders with hyperactivity and behavioural disorders. Finally, they can be employed in supplementary treatment in low-dose neuroleptic treatment.

The present invention had the object of finding further valuable pharmaceutical uses for the above-mentioned N-(indolecarbonyl)piperazine derivatives.

Although the use of these compounds for the treatment of sleep disorders and sleep apnoea is disclosed in WO 01/07435; it has, however, surprisingly now been found that they have—in contrast to conventional sleeping drugs—the pharmacologically important ability to extend both components of sleep, i.e. non-REM sleep (including slow-wave components thereof) and REM sleep.

Many people suffer from sleep disorders, which may on the one hand be a symptom of a disease, but on the other hand may also represent an independent syndrome. Thirty per cent of adults suffer from sleep disorders. Sleep disorders can manifest themselves in various ways:

Difficulties in falling asleep are characterised by the length of time a person needs to fall asleep. If this time is more than thirty minutes, the expression difficulties in falling asleep can be used. The person concerned then often lies awake for long periods, which in extreme cases can even last for hours.

If a patient suffers from premature awakening, the expression difficulties in staying asleep is used. However, this is only the case if the awakening occurs within six hours three times a week. The sleep is then often described as superficial and non-refreshing.

The expression premature awakening is used if the person concerned frequently wakes up much too early and then cannot fall asleep again.

The sleep of humans and many mammals, such as, for example, also rodents, can be divided roughly into the two stages of REM (=rapid eye movement) and non-REM, which occur alternately a number of times during sleep. As the name suggests, the eyes move rapidly in the eye sockets under the closed lids in the REM phase. This phase is the most intensive dreaming phase in humans. In non-REM sleep, a distinction is made between 4 stages, of which stages 3 and 4 are referred to as “slow-wave sleep”.

In order to achieve maximum refreshment during sleep, optimum sleep architecture is important, i.e. a balanced ratio between the two sleep phases. The total duration of sleep should be divided into the individual sleep stages as follows:

| | |
|---|---|
| non-REM stage 1: | 5% |
| non-REM stage 2: | 50% |
| non-REM stage 3 and 4: | 20% |
| REM: | 25% |

Whereas standard sleeping drugs merely extend the duration of non-REM sleep, with the duration of REM sleep remaining unchanged or even being reduced, the compounds according to the invention also increase the duration of REM sleep, which results in improved sleep architecture. By contrast, products on the market—such as, for example, triazolam, zolpidem or zoplicon—even shorten REM sleep.

It has already been known for some time that non-REM sleep (in particular the slow-wave components) in rats (Dugovic and Wauquier, Eur. J. Pharmacol. 137, 145-6, 1987) and also in humans (van Laar et al., Psychopharmacology (Berlin). 154, 189-97, 2001) is extended by 5-HT₂ receptor antagonists. However, it was unclear which receptor sub-type is responsible for this effect. Initially, the 5-HT_{2C} receptor was favoured (Sharpley et al., Neuropharmacology 33, 467-71, 1994).

Later, WO 00/12090 disclosed a selective antagonist of the 5-HT_{2A} receptor, R-(+)-alpha-(2,3-dimethoxyphenyl)-1-(2-(4-fluorophenyl)ethyl)-4-piperidinemethanol, which is suitable, inter alia, for the treatment of sleep disorders, effecting, in particular, an extension of slow-wave phases 3 and 4 of non-REM sleep.

By contrast, it has been reported that although non-selective 5-HT_{2A} antagonists, such as nefazodone, extend REM sleep, the slow-wave components of non-REM sleep remain unchanged (Sharpley and Cowen, Biol. Psychiatry 37, 85-98, 1995).

Although in thalidomide, which earlier used to be marketed under the name Contergan, a sleeping drug is known which likewise extends both sleep phases, this substance is not, however, a 5-HT₂ receptor antagonist.

At the present point in time, no antagonist of the 5-HT₂ receptors is known which is capable of extending both non-REM sleep and REM sleep. With the present invention, a novel active principle has thus been found which opens up novel possibilities for extending sleep and thus novel forms of therapy of sleep disorders.

Use is preferably made here of the following compounds, which are characterised in greater detail in WO 01/07435—where appropriate in the form of one of the salts thereof:

(1H-indol-4-yl)-(4-phenethylpiperazin-1-yl)methanone, (1H-indol-4-yl)-[4-(4-fluorophenethyl)piperazin-1-yl]methanone, (1H-indol-4-yl)-[4-(2,5-dichlorothiophen-3-ylethyl)piperazin-1-yl]methanone, (3-formyl-1H-indol-5-yl)-[4-(4-fluorophenethyl)piperazin-1-yl]methanone, (1H-indol-6-yl)-[4-(4-fluorophenethyl)piperazin-1-yl]methanone, (1H-indol-6-yl)-[4-(thiophen-2-ylethyl)piperazin-1-yl]methanone, hydrochloride, F. (1H-indol-6-yl)-[4-(2,5-dichlorothiophen-3-ylethyl)piperazin-1-yl]-methanone,

(3-cyano-1H-indol-6-yl)-[4-(4-fluorophenethyl)piperazin-1-yl]methanone, (1H-indol-7-yl)-(4-phenethylpiperazin-1-yl)methanone, (1H-indol-7-yl)-[4-(4-fluorophenethyl)piperazin-1-yl]methanone, (1H-indol-7-yl)-[4-(5-chlorothiophen-2-ylethyl)piperazin-1-yl]methanone, (3-formyl-1H-indol-7-yl)-[4-(4-fluorophenethyl)piperazin-1-yl]methanone, (3-cyano-1H-indol-7-yl)-[4-(4-fluorophenethyl)piperazin-1-yl]methanone, (2,3-dimethyl-1H-indol-7-yl)-[4-(4-fluorophenethyl)piperazin-1-yl]methanone,

(6,7,8,9-tetrahydro-5H-carbazol-3-yl)-(4-phenethylpiperazin-1-yl)methanone,

(3-formyl-1H-indol-6-yl)-[4-(4-fluorophenethyl)piperazin-1-yl]methanone, (1H-indol-6-yl)-[4-(5-chlorothiophen-2-ylethyl)piperazin-1-yl]methanone, (1H-indol-4-yl)-[4-(5-chlorothiophen-2-ylethyl)piperazin-1-yl]methanone, (3-cyano-1H-indol-5-yl)-[4-(4-fluorophenethyl)piperazin-1-yl]methanone, (3-cyano-1H-indol-7-yl)-[4-(naphth-2-ylethyl)piperazin-1-yl]methanone, (3-cyano-1H-indol-4-yl)-[4-(4-fluorophenethyl)piperazin-1-yl]methanone, (3-cyano-1H-indol-4-yl)-[4-(2-fluorophenethyl)piperazin-1-yl]methanone, (3-cyano-1H-indol-7-yl)-[4-(2-fluorophenethyl)piperazin-1-yl]methanone, (3-aminocarbonyl-1H-indol-7-yl)-[4-(4-fluorophenethyl)piperazin-1-yl]-methanone,

(3-cyano-1H-indol-7-yl)-[4-(4-fluorophenethyl)piperazin-1-yl]methanone, (3-cyano-1H-indol-7-yl)-[4-(5-chlorothiophen-2-ylethyl)piperazin-1-yl]-methanone, (3-cyano-1H-indol-7-yl)-(4-phenethylpiperazin-1-yl)methanone, (3-cyano-1H-indol-7-yl)-[4-(2,4-difluorophenethyl)piperazin-1-yl]methanone.

For the purposes of the invention, particular preference is given to the use of (3-cyano-1H-indol-7-yl)-[4-(4-fluorophenethyl)piperazin-1-yl]methanone and (3-aminocarbonyl-1H-indol-7-yl)-[4-(4-fluorophenethyl)piperazin-1-yl]-methanone.

Very particular preference is given to (3-cyano-1H-indol-7-yl)-[4-(4-fluoro-phenethyl)piperazin-1-yl]methanone.

The present invention therefore relates to the use of 5-HT₂ receptor antagonists, in particular 5-HT_{2A} receptor antagonists, for the preparation of a medicament for extending both non-REM sleep and REM sleep.

In this connection, it has been found that the N-(indolecarbonyl)piperazine derivatives according to the invention are particularly suitable for the treatment of difficulties in falling asleep and staying asleep and premature awakening in the morning.

The present invention therefore furthermore relates to the use of 5-HT₂ receptor antagonists, in particular 5-HT_{2A} receptor antagonists, for the preparation of a medicament for the treatment of difficulties in falling asleep and staying asleep and premature awakening in the morning.

The invention furthermore relates to the use of 5-HT₂ receptor antagonists for the preparation of a pharmaceutical preparation comprising the active ingredient according to the invention and optionally excipients and/or adjuvants and optionally further active ingredients.

The medicaments here can be converted into a suitable dosage form together with at least one solid, liquid and/or semi-liquid excipient or adjuvant and optionally in combination with one or more further active ingredient(s).

In the sleep therapy according to the invention, the 5-HT₂ receptor antagonists are generally administered analogously to known preparations, preferably in doses of between about 0.1 and 500 mg, in particular between 5 and 300 mg, per dosage unit. The daily dose is preferably between about 0.01 and 250 mg/kg, in particular between 0.02 and 100 mg/kg, of body weight.

The 5-HT₂ receptor antagonists are preferably administered here in doses of between about 1 and 500 mg, in particular between 5 and 100 mg, per dosage unit. The daily dose is preferably between about 0.02 and 10 mg/kg of body weight. However, the specific dose for each particular patient depends on a very wide variety of factors, for example on the efficacy of the specific compound employed, on the age, body weight, general state of health, sex, on the diet, on the time and method of administration, on the excretion rate, medicament combination and severity of the particular disease to which the therapy applies. Oral administration is preferred.

The 5-HT₂ receptor antagonists may also be employed together with other active ingredients, in particular other sleeping drugs, in the treatment of the diseases mentioned.

The invention therefore also relates to the use of 5-HT₂ receptor antagonists in combination with one or more further sleeping drugs in the sleep therapy described above.

Specific instructions for the synthesis of the 5-HT receptor-antagonistic N-(indolecarbonyl)piperazine derivatives described here are given in WO 01/07435.

The pharmaceutical preparations according to the invention can be employed as medicaments in human and veterinary medicine. Suitable excipients are organic or inorganic substances which are suitable for enteral (for example oral), parenteral or topical administration and do not react with the novel compounds, for example water, vegetable oils, benzyl alcohols, polyethylene glycols, gelatine, carbohydrates, such as lactose or starch, magnesium stearate, talc, Vaseline. Suitable for enteral administration are, in particular, tablets, coated tablets, capsules, syrups, juices, drops or suppositories, suitable for parenteral administration are solutions, preferably oil-based or aqueous solutions, furthermore suspensions, emulsions or implants, and suitable for topical application are ointments, creams or powders. The novel compounds may also be lyophilised and the resultant lyophilisates used, for example, for the preparation of injection preparations.

The preparations indicated may be sterilised and/or comprise adjuvants, such as lubricants, preservatives, stabilisers and/or wetting agents, emulsifiers, salts for modifying the osmotic pressure, buffer substances, dyes, flavours and/or aroma substances. They can, if desired, also comprise one or more further active ingredients, for example one or more vitamins.

The examples below relate to pharmaceutical preparations:

### EXAMPLE A1

### Injection Vials

A solution of 100 g of an active ingredient according to the invention and 5 g of disodium hydrogenphosphate in 3 l of bidistilled water is adjusted to pH 6.5 using 2N hydrochloric acid, sterile filtered, transferred into injection vials, lyophilised and sealed under sterile conditions. Each injection vial contains 5 mg of active ingredient.

### EXAMPLE A2

### Suppositories

A mixture of 20 g of an active ingredient according to the invention is melted with 100 g of soya lecithin and 1400 g of cocoa butter, poured into moulds and allowed to cool. Each suppository contains 20 mg of active ingredient.

### EXAMPLE A3

### Solution

A solution is prepared from 1 g of an active ingredient according to the invention, 9.38 g of NaH₂PO₄×2 H₂O, 28.48 g of NaH₂PO₄×12 H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE A4

### Ointment

500 mg of an active ingredient according to the invention are mixed with 99.5 g of Vaseline under aseptic conditions.

### EXAMPLE A5

### Tablets

A mixture of 1 kg of an active ingredient according to the invention, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### EXAMPLE A6

### Coated Tablets

Tablets are pressed analogously to Example E and subsequently coated in a conventional manner with a coating of sucrose, potato starch, talc, tragacanth and dye.

### EXAMPLE A7

### Capsules

2 kg of an active ingredient according to the invention are introduced into hard gelatine capsules in a conventional manner in such a way that each capsule contains 20 mg of the active ingredient.

### EXAMPLE A8

### Ampoules

A solution of 1 kg of an active ingredient according to the invention in 60 l of bidistilled water is transferred into ampoules, lyophilised under aseptic conditions and sealed under sterile conditions. Each ampoule contains 10 mg of active ingredient.

The action of the 5-HT₂ receptor-antagonistic N-(indolecarbonyl)piperazine derivatives according to the invention manifests itself as follows, as described using the example of (3-cyano-1H-indol-7-yl)-[4-(4-fluorophenethyl)piperazin-1-yl]methanone:

In experiments in which the brain waves of rats were recorded over 6 hours during the dark phase, the inventors of the present patent application have found that (3-cyano-1H-indol-7-yl)-[4-(4-fluorophenethyl)piperazin-1- yl]methanone at a dose of 3 mg/kg per os causes a maximum increase in non-REM sleep of about 5 minutes per hour, whereas the average increase is about 4 min/h.

The comparative substance triazolam, by contrast, extends non-REM sleep by 2 min/h at a dose of 0.1 mg/kg and by 6.5 min/h at a dose of 0.4 mg/kg, which corresponds to the maximum effect in triazolam.

Under the same conditions, zolpidem extends non-REM sleep by 5 min/h at 5 mg/kg and by 7 min/h at 10 mg/kg. Zoplicon (2.5-5 mg/kg) exhibits a comparable effect.

(3-Cyano-1H-indol-7-yl)-[4-(4-fluorophenethyl)piperazin-1-yl]methanone is thus comparable with the reference sleeping drugs in its ability to extend non-REM sleep.

However, there is an important difference between the compound according to the invention and the reference sleeping drugs with respect to their action on REM sleep. The standard sleeping drugs shorten this stage of sleep: triazolam (0.1-1.6 mg/kg) by 0.3 to 2.1 min/h, zolpidem (5-10 mg/kg) and zopiclon (2.5-5 mg/kg) by 0.3 to 1.6 min/h (the values relate to recording on rats for 6 hours during the dark phase). These differences emanate from a reduction in the duration of the individual REM phases (triazolam) or from a reduction in the number of these phases (zolpidem/zopiclon). (3-Cyano-1H-indol-7-yl)-[4-(4-fluorophenethyl)piperazin-1-yl]-methanone, by contrast, extends REM sleep by an average of 0.8 min/h and with a maximum of 2 min/h. This results essentially from the increase in the number of REM episodes.

This property of (3-cyano-1H-indol-7-yl)-[4-(4-fluorophenethyl)piperazin-1-yl]methanone is thus unique and opens up novel possibilities for extending sleep, in particular in the treatment of difficulties in falling asleep and staying asleep and premature awakening in the morning.

The above-described efficacy of (3-cyano-1H-indol-7-yl)-[4-(4-fluorophen-ethyl)piperazin-1-yl]methanone in the treatment of the sleep disorders according to the invention can be determined in vivo as follows.

### EXAMPLE B

### Treatment of Rats with (3-cyano-1H-indol-7-yl)-[4-(4-fluoro- phenethyl)piperazin-1-yl]methanone, hydrochloride

In order to measure the brain waves, EEG electrodes are implanted into the brain of anaesthetised rats. After a recovery time of 15 days, these electrodes are connected to an amplifier via a flexible cable, and the brain waves of the non-anaesthetised animals are recorded over 12 hours. (3-Cyano-1H-indol-7-yl)-[4-(4-fluorophenethyl)piperazin-1-yl]methanone is dissolved in advance in a concentration of 0.1 ml/100 g of peanut oil. This solution (compound) or, for comparison, merely the solvent (vehicle) is administered orally to the test animals in a dose of 3 mg/kg. From the filtered and amplified brain-wave signals, the sleep stages are evaluated via Fourier spectral analysis including certain criteria. The REM and non-REM sleep stages can be identified with reference to the patterns. The experimental results are shown in Tables 1 (effect of the substance) and 2 (significances of the measurement values). It becomes clear that (3-cyano-1H-indol-7-yl)-[4-(4-fluorophenethyl)piperazin-1-yl]methanone results in a significant extension both of non-REM sleep and of REM sleep and that these extensions are significant.

TABLE 1
Effect of (3-cyano-1H-indol-7-yl)-[4-(4-fluorophenethyl)piperazin-1-yl]
methanone on various sleep parameters of rats (average ± standard error).

| | REM sleep | | NREM sleep | | Wakefulness | |
|---|---|---|---|---|---|---|
| | Vehicle | Compound | Vehicle | Compound | Vehicle | Compound |
| Total Time (min) | 35.3 ± 2.2 | 45.3 ± 3.8 | 185.8 ± 7.9 | 232.8 ± 13.1 | 497.6 ± 8.5 | 440.6 ± 15.1 |
| Episode Duration | 79.6 ± 3.6 | 93.1 ± 6.6 | 147.0 ± 5.4 | 184.8 ± 9.5 | 457.2 ± 31.2 | 422.5 ± 29.3 |
| (sec) | | | | | | |
| REM Latency (min) | 3.9 ± 0.1 | 5.2 ± 0.3 | | | | |
| Inter-REM interval | 29.6 ± 1.7 | 28.8 ± 3.0 | | | | |
| (min) | | | | | | |

Total Time: Time over the measurement time period spent in the respective sleep stages
Episode Duration: Mean duration of an episode of the respective sleep stage
REM Latency: Period from the beginning of sleep to entry into the first REM phase
Inter-REM interval: Average time between the intervals in the REM stage
Compound: Label for the animals which have received the test substance.
Vehicle: Label for the animals which have only received the solvent.
Wakefulness: State of being awake

This experiment is a crossover study. This means that one and the same animal receives either first solvent (vehicle) and then, after a waiting time of one week, the test substance (3-cyano-1H-indol-7-yl)-[4-(4-fluorophenethyl)piperazin-1-yl]methanone (compound), or the administration is carried out in the reverse sequence.

TABLE 2
Significance of the values from Table 1.

| | Precise p values from |
|---|---|
| | ANOVA for measurement |
| | value repetitions |
| REM time | 0.04 |
| REM duration | 0.1 (n.s.) |
| NREM time | 0.01 |
| NREM duration | 0.003 |
| Wakefulness time | 0.005 |
| Wakefulness duration | 0.5 (n.s.) |
| REM latency | 0.0002 |
| Inter-REM interval | 0.8 (n.s.) |

(n.s.) the respective measurement values from Table 1 are not significant

The measured values after vehicle or compound administration are compared with one another using the statistical method of analysis of variance (ANOVA). The p value is a statistical measure of the probability that a difference occurs by chance between the measurement values or is caused by the substance administration. According to international standards, a p value of below 0.05 is regarded as “significant”.

It is clear from FIG. 1 that in particular stages 3 and 4, which are regarded as slow-wave sleep, are extended in non-REM sleep. The curve shows the effect of (3-cyano-1H-indol-7-yl)-[4-(4-fluorophenethyl)piperazin-1-yl]methanone on the relative delta power in the rat EEG, expressed as the difference from the control level (dotted zero line), as a function of the time of day. The term delta power or delta waves denotes the “slow” waves recorded in the EEG which are characteristic of slow-wave sleep stages. For each rat, the hour average after solvent (vehicle) treatment was firstly determined and subtracted from the value after substance treatment. The relative delta power is significantly increased overall.

1. Use of 5-HT₂ receptor antagonists and physiologically acceptable salts and solvates thereof for the preparation of a medicament for extending both non-REM sleep and REM sleep.

2. Use according to claim 1, characterised in that the 5-HT₂ receptor antagonists are of the 5-HT2a sub-type.

3. Use of 5-HT2a receptor antagonists according to claim 2, selected from a group consisting of
(a) (3-aminocarbonyl-1H-indol-7-yl)-[4-(4-fluorophenethyl)piperazin-1-yl]methanone,
(b) (3-cyano-1H-indol-7-yl)-[4-(4-fluorophenethyl)piperazin-1-yl]methanone and physiologically acceptable salts and solvates thereof for the preparation of a medicament for extending both non-REM sleep and REM sleep.

4. Use of (3-cyano-1H-indol-7-yl)-[4-(4- fluorophenethyl)piperazin-1-yl]methanone and physiologically acceptable salts and solvates thereof for the preparation of a medicament for extending both non-REM sleep and REM sleep.

5. Use of 5-HT₂ receptor antagonists and physiologically acceptable salts and solvates thereof according to claim 1 for the preparation of a medicament for the treatment of difficulties in falling asleep and staying asleep and premature awakening in the morning.

6. Use of 5-HT₂ receptor antagonists and physiologically acceptable salts and solvates thereof according to claim 1 in combination with one or more further sleeping drugs.
